(19) United States
(12) Patent Application Publication
(10) Pub. No.: US 2016/0317129 A1
(43) Pub. Date: Nov. 3, 2016

(54) SYSTEM AND METHOD FOR ULTRASOUND AND COMPUTED TOMOGRAPHY IMAGE REGISTRATION FOR SONOTHROMBOLYSIS TREATMENT

(71) Applicant: KONINKLIJKE PHILIPS N.V., Eindhoven (NL)

(72) Inventors: Ralf Seip, Carmel, NY (US); Sandeep M. Dalal, Cortlandt Manor, NY (US)

(21) Appl. No.: 15/104,577

(22) PCT Filed: Dec. 8, 2014
(86) PCT No.: PCT/IB2014/066684

§ 371 Date: Jun. 15, 2016

Related U.S. Application Data
(60) Provisional application No. 61/917,755, filed on Dec. 18, 2013.

(51) Int. Cl. A61B 8/08; A61N 7/00; A61B 6/00; A61B 6/03

(52) U.S. Cl. CPC A61B 8/5261; A61B 6/032; A61B 8/488; A61B 8/0808; A61N 7/00; A61B 8/483; A61B 8/0891; A61B 6/504; A61N 2007/0052; A61N 2007/0004

(57) ABSTRACT

A method and system for treating a target area of a patient, for example an area of the brain which includes an occlusion: employ an ultrasound imaging apparatus to produce an ultrasound image of a region of a subject; register the ultrasound image to a computed tomography (CT) image dataset; identify in the ultrasound image a location of a target area via a marker of the target area produced from the CT image dataset; verify the location of the target area with the ultrasound imaging apparatus; and provide sonothrombolysis treatment to the target area while monitoring the target area with the ultrasound imaging apparatus.

### TECHNICAL FIELD

This invention relates to medical acoustic (e.g., ultrasound) systems and, in particular, to ultrasound systems which perform therapy for stroke victims.

### BACKGROUND AND SUMMARY

Ischemic stroke is a debilitating disorder. The blockage of the flow of blood to the brain can rapidly result in paralysis or death. Attempts to achieve recanalization through thrombolytic drug therapy such as treatment with tissue plasminogen activator (tPA) has been reported to cause symptomatic intracerebral hemorrhage in a number of cases. Advances in the diagnosis and treatment of this crippling affliction are the subject of continuing medical research.

Use of ultrasound waves is an emerging non-invasive stroke treatment modality which is applied to help lyse blood clots causing vascular occlusion. In particular, sonothrombolysis (STL) treatments utilizing ultrasound (US) (targeting the clot) in conjunction with microbubbles for clot dissolution and vessel recanalization are currently being investigated as strong treatment alternatives for acute stroke patients. In STL treatments, ultrasound pulses are delivered through the skull temporal bone, targeted at the clot that causes the occlusion. Microbubbles, an ultrasound contrast agent, also form an integral part of the STL treatment, as their mechanical oscillation at the clot site due to the applied ultrasound energy has been shown to dissolve the clot over time and achieve vessel recanalization for acute ischemic stroke treatment. One of the advantages of STL treatments is that they can be performed non-invasively and without the use of drugs (such as t-PA, or tissue plasminogen activator, a common “clotbusting” drug), which carry with them significant restrictions to their use, and overall low treatment success.

However, such treatments require the delivery of a specific ultrasound dose targeted at the clot. One challenge associated with STL treatments is that the ultrasound delivery devices currently being evaluated in clinical trials for sonothrombolysis stroke therapy lack an imaging function, which would enable them to also be used to localize the clot position within the brain. This leads to overtreatment (i.e. a larger region (which hopefully) contains the clot must be treated to bring about recanalization), or no treatment at all (as a region is treated that does not even contain the clot), and provides limited to no treatment feedback during the administration of the ultrasound energy.

Accordingly, it would be desirable to provide a method and system for sonothrombolysis treatment or therapy with imaging that identifies the location of the target area to be treated in real-time to guide the sonothrombolysis treatment ultrasound energy to the target area.

In one aspect of the invention, a method comprises: receiving a computed tomography (CT) image dataset produced by a computed tomography system; employing an ultrasound imaging apparatus to produce an ultrasound image of a region of a subject including a target area to be treated with sonothrombolysis treatment; generating one or more fiducial markers for the ultrasound image, wherein the one or more fiducial markers identify a recognizable feature of the subject; a processor employing the one or more fiducial markers for the ultrasound image, and one or more corresponding fiducial markers for the CT image dataset, to register the ultrasound image with the CT image dataset including a marker identifying the location of the target area in the CT image dataset; superimposing the marker identifying the location of the target area in the CT image dataset with the ultrasound image to produce a superimposed ultrasound image, and displaying the superimposed ultrasound image; verifying the location of the target area with the ultrasound imaging apparatus; and applying the sonothrombolysis treatment to the verified location of the target area.

In some embodiments, the region of the subject includes at least a portion of the subject's head, and wherein the target area corresponds to an area of an occlusion in the subject's brain.

In some versions of these embodiments, the one or more fiducial markers include one or more markers identifying at least one of: an outline of at least a portion of the subject's skull bone, a location of the subject's contralateral skull bone, a location of the subject's contralateral skull bone, a location of the subject's brain stem, a location of the subject's temporal bone, and one or more corresponding cerebral vessels of the subject.

In some versions of these embodiments, the method further comprises employing the ultrasound imaging apparatus to provide real time imaging of the target area while applying the sonothrombolysis treatment to the target area.

In some versions of these embodiments, the method further comprises determining from the real-time imaging whether the occlusion has been cleared.

In some versions of these embodiments, the method further comprises determining from the real-time imaging whether blood flow has been restored in the area of the occlusion.

In some versions of these embodiments, the method further comprises stopping the sonothrombolysis treatment once it has been determined that blood flow has been restored in the area of the occlusion.

In some versions of these embodiments, the method further comprises one or more processors ascertaining one or more values for one or more corresponding imaging parameters of the ultrasound imaging apparatus and treatment parameters of the sonothrombolysis treatment based on the location of the target area, so as to obtain desired imaging of the target area.

In some versions of these embodiments, employing the ultrasound imaging apparatus to provide real time imaging of the target area comprises employing a Doppler based algorithm to process signals received by the ultrasound imaging apparatus from the target area.

In some versions of these embodiments, applying the sonothrombolysis treatment to the verified location of the target area includes: positioning a headset on the subject's head, wherein the headset includes at least one ultrasound transducer array configured to supply ultrasound treatment to an adjustable treatment region; and automatically adjusting at least one of a position and an orientation of the ultrasound transducer array to cause the treatment region to match the target area.

In some embodiments, the method further includes generating the one or more fiducial markers for the CT image dataset.

In another aspect of the invention, a system comprises: a sonothrombolysis treatment apparatus; and an ultrasound imaging apparatus. The system includes one or more processors configured to: control the ultrasound imaging apparatus to produce an ultrasound image of a region of a subject, identify in the ultrasound image a location of a target area via a marker for the target area produced from a computed tomography (CT) image dataset, and control the sonothrombolysis treatment apparatus to provide sonothrombolysis treatment to the target area while controlling the ultrasound imaging apparatus to image the target area.

In some embodiments, the ultrasound apparatus and the sonothrombolysis treatment apparatus may share one or more common components, such as a processor, memory, beamformer(s), ultrasound array(s), etc.

In some embodiments, the sonothrombolysis treatment apparatus includes a headset including at least one ultrasound transducer array configured to supply ultrasound treatment to an adjustable treatment region and to image the treatment region, and wherein controlling the sonothrombolysis treatment apparatus to provide sonothrombolysis treatment to the target area includes automatically adjusting at least one of a position and an orientation of the ultrasound transducer array to cause the treatment region to match the target area.

In some embodiments, the one or more processors are configured to ascertain at least one of one or more values for one or more imaging parameters of the ultrasound imaging apparatus and treatment parameters of the sonothrombolysis treatment apparatus based on the location of the target area so as to obtain at least one of a desired imaging and a desired treatment of the target area.

In some versions of these embodiments, the one or more processors are configured to automatically adjust at least one of the one or more imaging parameters of the ultrasound imaging apparatus and the treatment parameters of the sonothrombolysis treatment apparatus to have the one or more ascertained values.

In some versions of these embodiments, the one or more processors are configured to indicate to a user of the system the one or more determined values for the one or more corresponding imaging parameters of the ultrasound imaging apparatus.

In some embodiments, the ultrasound imaging apparatus includes a Doppler mode for processing images of the target area.

In yet another aspect of the invention, a method comprises: employing an ultrasound imaging apparatus to produce an ultrasound image of a region of a subject; identifying in the ultrasound image a location of a target area via a marker of the target area produced from a computed tomography (CT) image dataset; verifying the location of the target area with the ultrasound imaging apparatus; and providing sonothrombolysis treatment to the target area while monitoring the target area with the ultrasound imaging apparatus.

In some embodiments, providing sonothrombolysis treatment to the target area includes automatically adjusting at least one of: a position of a headset positioned on the subject, an orientation of the ultrasound transducer array of a headset positioned on the subject, and one or more treatment parameters, to cause a treatment region of the ultrasound transducer array to match the target area.

In some embodiments, the method further comprises automatically ascertaining one or more values for one or more corresponding imaging parameters of the ultrasound imaging apparatus and the one or more treatment parameters based on the location of the target area so as to obtain desired imaging of the target area; and adjusting at least one of the one or more imaging parameters and one or more treatment parameters to have the one or more determined values.

In some versions of these embodiments, the method further comprises ending the treatment based on a presence or amount of blood flow detected while monitoring the target area with the ultrasound imaging apparatus.

### BRIEF DESCRIPTION OF THE DRAWINGS

FIG. 1 shows a cranial angiographic computed tomography (CT) image.

FIG. 2 illustrates stereotactic registration (ruler-based) between a CT image dataset and a subject's head, and subsequent therapy ultrasound transducer positioning and alignment.

FIG. 3 is diagram illustrating one embodiment of an arrangement for generating a computed tomography (CT) image and registering the CT image with a sonothrombolysis treatment system.

FIG. 4 shows an example of an angiographic computed tomography (CT) image locating a clot in a major cerebral artery.

FIG. 5 illustrates one embodiment of a headset for a sonothrombolysis treatment apparatus.

FIG. 6 illustrates an example of a side-by-side registered view of a live ultrasound image and a CT image during the application of sonothrombolysis treatment to an occlusion or blood clot identified on a CT image which is registered with an ultrasound image.

FIG. 7 illustrates one example embodiment of a CT imaging system.

FIG. 8 is a functional block diagram of one embodiment of a sonothrombolysis treatment system.

FIG. 9 is a flowchart of one embodiment of a method for sonothrombolysis treatment of a target area, in particular an area of a blood clot causing vascular occlusion.

### DETAILED DESCRIPTION

The present invention will now be described more fully hereinafter with reference to the accompanying drawings, in which preferred embodiments of the invention are shown. This invention may, however, be embodied in different forms and should not be construed as limited to the embodiments set forth herein. Rather, these embodiments are provided as teaching examples of the invention.

As described above, ultrasound delivery devices currently being evaluated in clinical trials for sonothrombolysis stroke therapy lack an imaging function, which would enable them to also be used to localize the clot position within the brain. This leads to overtreatment (i.e. a larger region (which hopefully) contains the clot must be treated to bring about recanalization), or no treatment at all (as a region is treated that does not even contain the clot), and provides limited to no treatment feedback during the administration of the ultrasound energy.

Ideally, a STL stroke treatment system would incorporate both an ultrasound imaging function for stroke diagnosis and clot position location, as well as an ultrasound therapy function for stroke treatment/vessel recanalization in a single device. This would have the advantage that both functions (diagnosis/imaging and treatment) could be co-registered and share the same coordinate system, making the step from clot identification to clot targeting via treatment planning a simple process. However, ultrasound by itself is not typically used to diagnose the stroke. For ultrasound to be effective for stroke diagnosis, it needs to be combined with ultrasound contrast agents, which significantly improve the ability to locate the vessel occlusion causing the stroke. Ultrasound contrast agents, however, are currently not indicated in the USA and several other countries to be used for stroke diagnosis.

Treatment planning and clot targeting under ultrasound guidance is thus challenging.

A final challenge is related to the current workflow and standard of care: CT (with contrast) is the de-facto ‘gold standard’ for stroke diagnosis and is well established, thus unlikely to be replaced for stroke diagnosis with other modalities (i.e. contrast ultrasound) easily and in the near future.

Until combined ultrasound imaging/treatment devices and procedures for STL are approved for clinical use and become available in the clinic, and ultrasound contrast agents are indicated for stroke diagnosis, alternate methods need to be put in place to still be able to utilize ultrasound for stroke treatment.

Systems and methods described below can accurately target the region of an occlusion with ultrasound to achieve vessel recanalization and thus treat the stroke, without the need to use ultrasound contrast agents to locate the target region, by obtaining diagnosis and targeting information from a separate computed tomography (CT) scan.

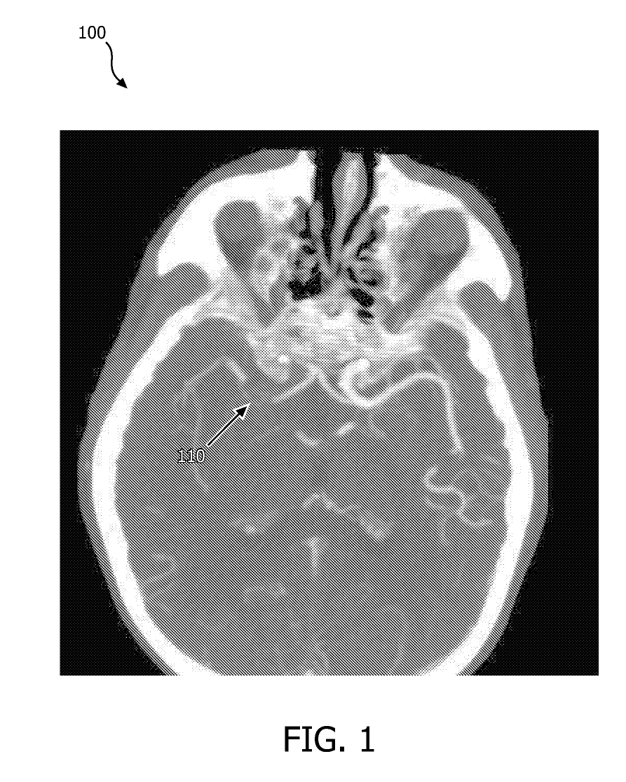

FIG. 1 shows a cranial angiographic computed tomography (CT) image 100 which indicates the presence of an occlusion or blood clot 110.

As noted above, embodiments of systems and methods described below employ a CT image dataset such as that represented by CT image 100 for guiding sonothrombolysis treatment to the area of the occlusion or blood clot 110.

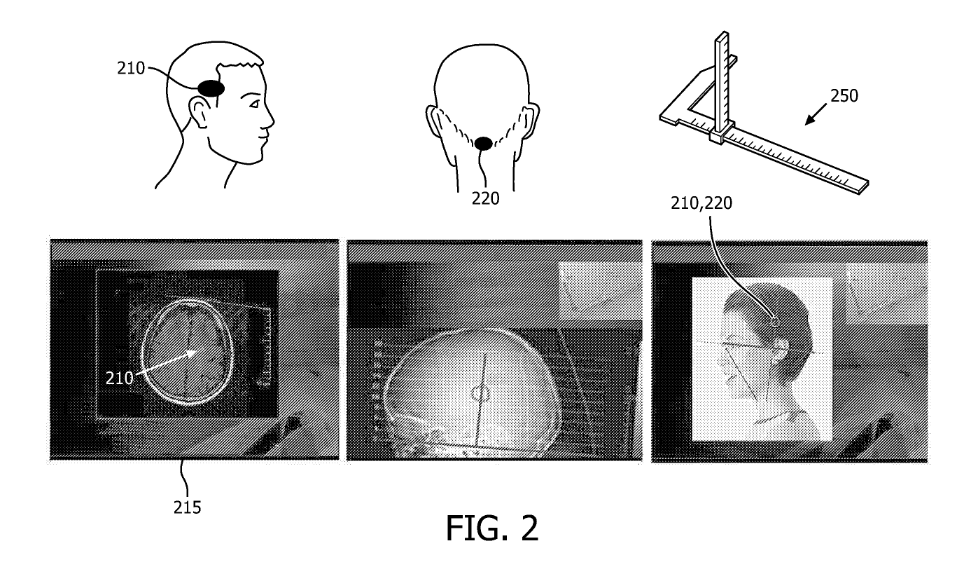

FIG. 2 illustrates one method which has been proposed for guiding sonothrombolysis treatment to the area of occlusion or blood clot 110. In particular, FIG. 2 illustrates a process of stereotactic (ruler-based) registration between a CT image dataset and a subject's head, and subsequent therapy ultrasound transducer positioning and alignment. As illustrated in FIG. 2, an occlusion or blood clot 210 is identified on a CT image 215, together with a reference point 220. The CT image is then marked up to indicate the distance from reference point 220 to occlusion or blood clot 210 in a coordinate system. Then, a ruler 250 is employed, together with the reference point of the patient's head, to measure off the distance to find the location of the occlusion or blood clot 210 in the patient's head.

It is apparent that the stereotactic method described above is not an optimum solution identifying the location of an occlusion or blood clot with respect to a patient's head, and even less so to guide sonothrombolysis treatment to the location.

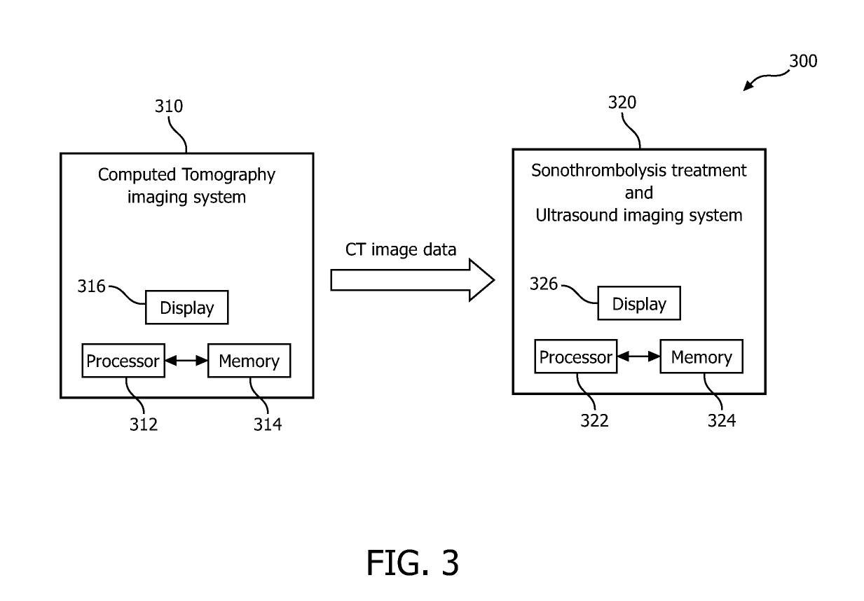

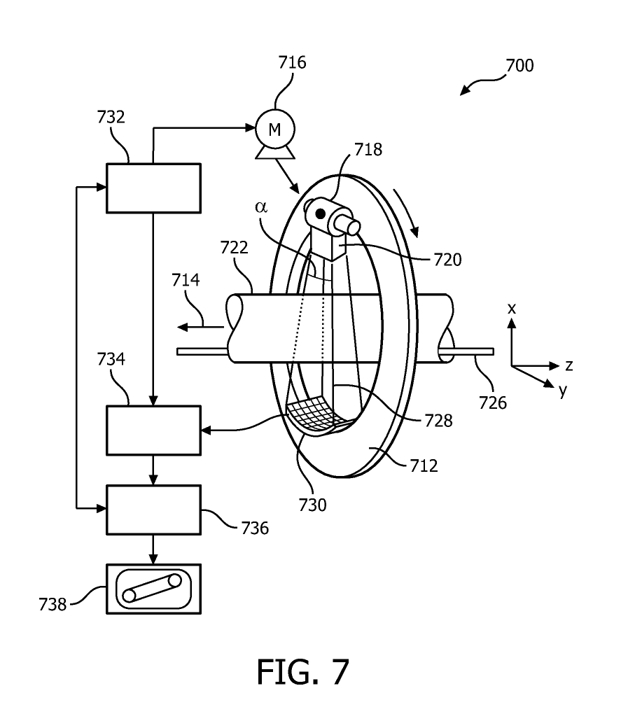

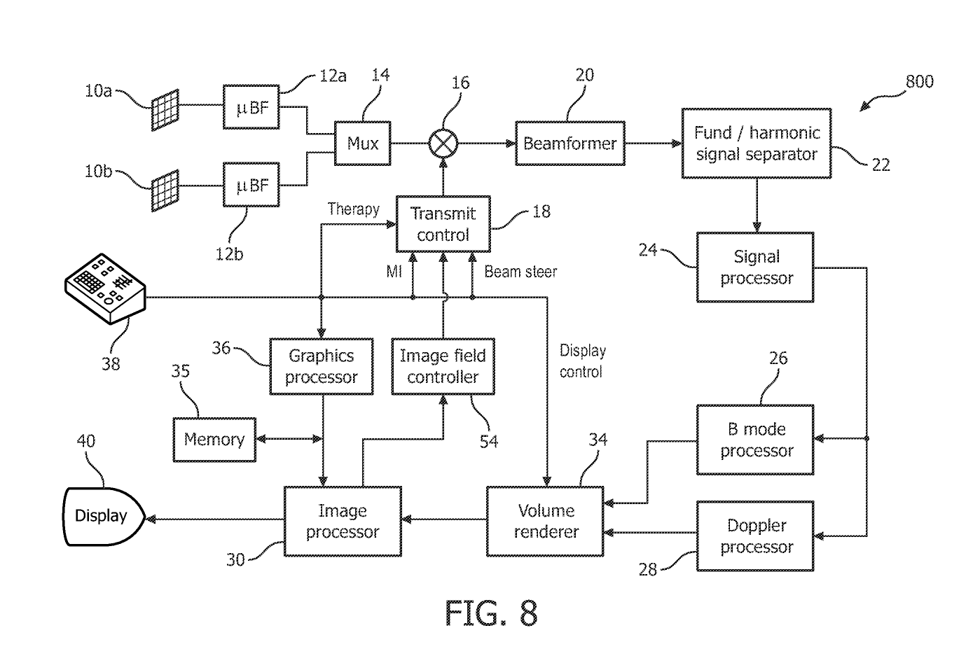

FIG. 3 is a diagram illustrating one embodiment of an arrangement 300 for generating a computed tomography (CT) image and registering the CT image with a sonothrombolysis treatment system. Arrangement 300 employs an a CT image dataset generated by a computed tomography (CT) imaging system 310 to assist the guidance of sonothrombolysis treatment from a sonothrombolysis treatment and ultrasound imaging system 320 to a target area of a subject where an occlusion or blood clot has been identified. Among other components, CT imaging system 310 includes one or more processors 312, associated memory 314, and a display device 316. Further details of one embodiment of CT imaging system 310 will be described below with respect to FIG. 7. Among other components, sonothrombolysis treatment and ultrasound imaging system 320 includes one or more processors 322, associated memory 324, and a display device 326. Further details of one embodiment of sonothrombolysis treatment and ultrasound imaging system 320 will be described below with respect to FIG. 8.

An example of a process of employing arrangement 300 for sonothrombolysis treatment will now be described.

In this example, CT imaging system 310 is employed for diagnosing the stroke (ischemic or hemorrhagic). If the stroke is ischemic, CT imaging system 310 employs perfusion or angiographic CT to determine the presence and location of the blood clot, the tissue core that is irreversibly infarcted, and the tissue that is potentially salvageable. CT imaging system 310 generates a CT image dataset (e.g., a 3D image dataset) which contains the CT images. One or more markers are added to the CT image dataset, identifying or highlighting the location(s) of the occlusion(s) or blood clot(s). In some embodiment, the CT image dataset may further also contain one or more fiducial markers which indicate the location of one or more features of the subject's anatomy which are readily identifiable in the CT image dataset and also in an ultrasound image. For example, one fiducial marker may be the temporal bone, i.e. the location within the skull bone corresponding to the most appropriate acoustic window, so as to minimize the attenuation of the therapeutic ultrasound pulses as they propagate through the skull. As used herein, the term “fiducial marker” refers to an object in the field of view of an imaging system which appears in an image produced by the imaging system and which may be employed as a point of reference or measure. In example embodiments disclosed herein, fiducial markers may include markers identifying: an outline of at least a portion of the subject's skull bone; a location of the subject's contralateral skull bone; a location of the subject's temporal bone; the brain stem; and one or more cerebral vessels of the subject, such as the middle cerebral artery (MCA) or the Circle of Willis. The use of other fiducial markers is contemplated. In some embodiments, the fiducial marker(s) and/or the markers identifying or highlighting the location(s) of the occlusion(s) or blood clot(s) with respect to the CT imaging dataset may be generated by a user or clinician via CT imaging system 310. In other embodiments, the CT imaging data generated by CT imaging system 310 may be transferred to a separate computer, workstation, or other data processing device, and the fiducial marker(s) in the CT dataset and/or the markers identifying or highlighting the location(s) of the occlusion(s) or blood clot(s) with respect to the CT imaging dataset may be generated by a user or clinician via that computer, workstation, or other data processing device. In still other embodiments, the fiducial marker(s) in the CT dataset and/or the markers identifying or highlighting the location(s) of the occlusion(s) or blood clot(s) with respect to the CT imaging dataset may be generated by a user or clinician via sonothrombolysis treatment and ultrasound imaging system 320.

The CT image dataset is transferred to sonothrombolysis treatment and ultrasound imaging system 320. Marker(s) identifying or highlighting the location(s) of the occlusion(s) or blood clot(s) and fiducial markers may be added (for example, by a clinician) to the CT image dataset via the CT imaging system 310, or by an intermediary data processing system not shown in FIG. 3, in which case the CT image dataset is transferred to sonothrombolysis treatment and ultrasound imaging system 320 together with the one or more markers identifying or highlighting the location(s) of the occlusion(s) or blood clot(s) and fiducial markers. In an alternative arrangement, the CT image dataset transferred from CT imaging system 310 to sonothrombolysis treatment and ultrasound imaging system 320, and marker(s) identifying or highlighting the location(s) of the occlusion(s) or blood clot(s) and fiducial markers may be added (for example, by a clinician) to the CT image dataset via sonothrombolysis treatment and ultrasound imaging system 320.

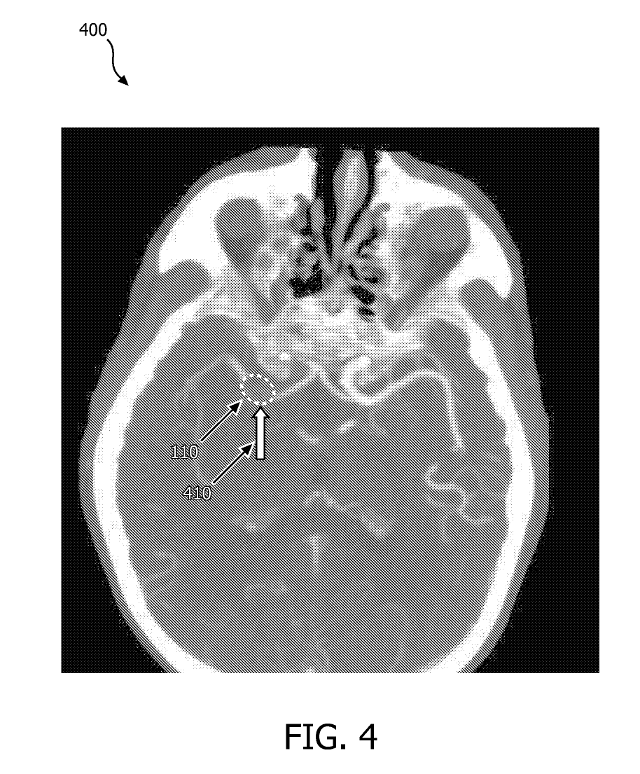

FIG. 4 shows an example of an angiographic computed tomography (CT) image 400 identifying the location of a clot in a major cerebral artery of a subject's brain. In particular, image 400 has been annotated or marked with a marker 410 which indicates the location of an occlusion or blood clot in the subject's brain.

Depending on the data input interfaces of the sonothrombolysis treatment and ultrasound imaging system 320, the CT image data may be transferred via wireless link, via a network (e.g., an intranet or internet), via a portable data storage medium such as a DVD or a Flash memory device, etc. In some embodiments, the CT image dataset may be transferred from CT imaging apparatus 310 to a network server and associated data storage device, and then transferred from the network server to sonothrombolysis treatment and ultrasound imaging system 320.

Sonothrombolysis treatment and ultrasound imaging system 320 may employ ultrasound for imaging the brain and skull without contrast, and also employ ultrasound to deliver the sonothrombolysis pulses for therapy delivery, for example via a probe/headset placed on the subject's head.

Example embodiments of a headset which may be employed which may be employed for sonothrombolysis treatment are disclosed in: U.S. Provisional Patent Application 61/906,973, filed on Nov. 21, 2013; U.S. Provisional Patent Application 61/716,007, filed on Oct. 19, 2013; U.S. Provisional Patent Application 61/865279, filed on Aug. 13, 2013; and International Patent Application PCT/IB2013/059268, filed on Oct. 10, 2013.

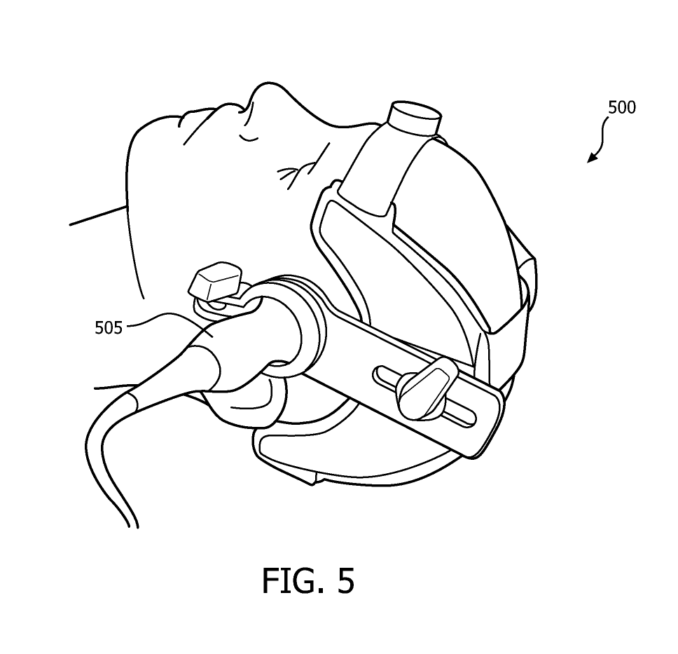

FIG. 5 illustrates one embodiment of a headset 500 for a sonothrombolysis treatment apparatus. Headset 500 includes at least one ultrasound transducer array 505 which may be employed for sonothrombolysis treatment and ultrasound imaging. Ultrasound therapy and imaging may be directed to an area of interest by controlling the operation of ultrasound transducer array(s) 505.

In operation, sonothrombolysis treatment and ultrasound imaging system 320 acquires 2D and/or 3D ultrasound images of the subject's head 10 in real-time, for example via headset 500. Sonothrombolysis treatment and ultrasound imaging system 320 combines the CT image dataset received from CT imaging system 310 with the real-time 2D and 3D ultrasound datasets for treatment planning, therapy delivery, and treatment monitoring.

Beneficially, to facilitate sonothrombolysis treatment to a desired target are where an occlusion or blood clot may be located, sonothrombolysis treatment and ultrasound imaging system 320 (e.g., processor 322 and memory 324) may execute a registration algorithm to register CT images of the CT image dataset with live ultrasound images.

In some embodiments, sonothrombolysis treatment and ultrasound imaging system 320 (e.g., processor 322 and memory 324) may execute an automatic registration algorithm which employs one or more fiducial markers in the CT image dataset and one or more corresponding fiducial markers in the ultrasound image for real-time registration. In some embodiments, sonothrombolysis treatment and ultrasound imaging system 320 (e.g., processor 322 and memory 324) may execute an image-fusion algorithm to present on display device 326 an overlay or a side-by-side registered view of the live ultrasound image and the CT image. Image-fusion may provide a common coordinate system for identifying target locations for therapy delivery.

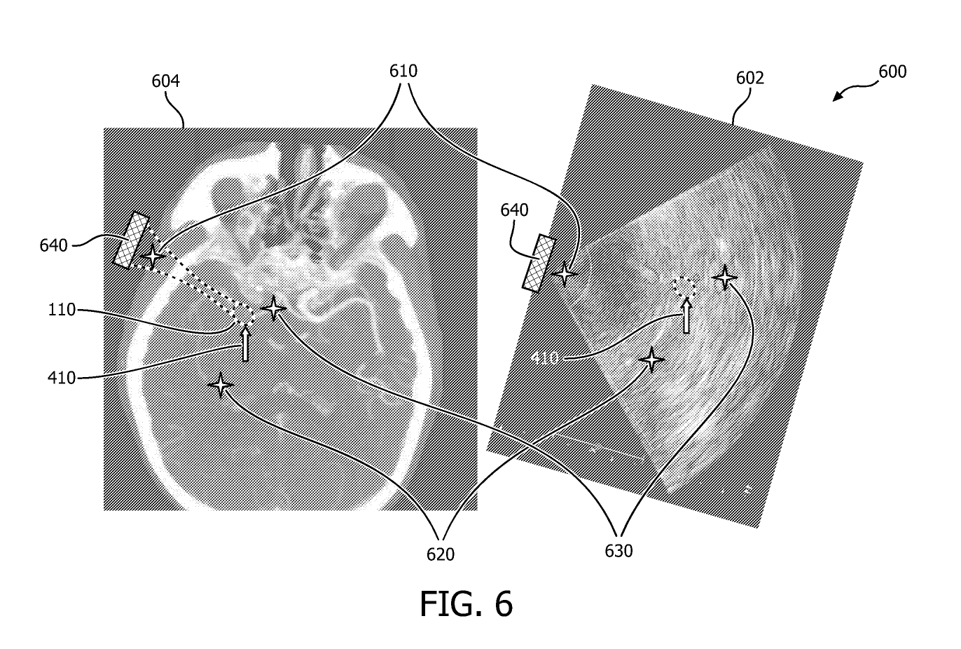

FIG. 6 illustrates an example of a side-by-side registered view 600 of a live ultrasound image 602 and a CT image 604 during the application of sonothrombolysis treatment to an occlusion or blood clot 110. Also shown in FIG. 6 are three different types of fiducial markers which may be employed alone or together to register ultrasound image 602 with CT image 604. The fiducial markers include: a first fiducial marker 610 corresponding to the location of the temporal bone in both ultrasound image 602 and CT image 604; a second fiducial marker 620 corresponding to the location of brain stem in both ultrasound image 602 and CT image 604; and a third fiducial marker 630 corresponding to the location of a particular blood vessel in both ultrasound image 602 and CT image 604. By use of the fiducial marker(s), ultrasound image 602 is registered with CT image 604, and marker 410 from CT image 604 is superimposed on ultrasound image 602 to identify the location of the target area which includes an occlusion or blood clot 110 and at which location sonothrombolysis treatment should be applied. With this information, a clinician is able to employ a sonothrombolysis treatment apparatus including an ultrasound transducer array 640 to accurately direct sonothrombolysis treatment to the target area including occlusion or blood clot 110.

Registration of the ultrasound images with the CT image dataset allows use of the CT image to enable sonothrombolysis treatment and ultrasound imaging system 320 to accomplish some of all of the following.

Sonothrombolysis treatment and ultrasound imaging system 320 may verify that the therapeutic ultrasound probe is correctly positioned on the temporal bone (region with lowest acoustic attenuation as determined from CT images), and provide re-positioning information should it not be positioned correctly.

Sonothrombolysis treatment and ultrasound imaging system 320 may verify that the therapeutic ultrasound probe is correctly oriented in the direction of the clot (clot identified via absence/stoppage of flow from CT contrast agent), and provide re-orientation information should it not be oriented correctly.

Sonothrombolysis treatment and ultrasound imaging system 320 may automatically adjust the ultrasound imaging parameters (depth, focal depth, gain, Doppler region, color Doppler window, etc.) based on the location, position, and region surrounding the clot, or provide a ‘best settings’ recommendation to the clinician, and further adjust the treatment parameters (power, treatment volume, etc.) based on the location, position, depth, etc. surrounding the clot, or provide a ‘best settings’ recommendation to the clinician.

Sonothrombolysis treatment and ultrasound imaging system 320 may highlight or superimpose the clot location and its extent as obtained from the CT dataset on the ultrasound images in real-time.

Sonothrombolysis treatment and ultrasound imaging system 320 may automatically define the target region/treatment window, in preparation for clinician review and initiation of therapy.

Sonothrombolysis treatment and ultrasound imaging system 320 may select a high-resolution, high-sensitivity, image compounding mode, or other specialized ultrasound imaging mode (that may otherwise be time/resource-intensive to implement) only in the region surrounding the clot, specifically designed to generate ultrasound images for clot detection that overcome or partially compensate for the absence of ultrasound contrast agents for clot location. This could be, for example, a Doppler-based algorithm that estimates and averages flow values over several heartbeats, in order to increase the sensitivity of the measurement and SNR of the data. Such modes would be resource-intensive to implement for the entire ultrasound field of view. Also beneficially, these modes may be combined with a probe or headset where the ultrasound probe positioning is controlled electronically, such as with a motorized probe positioning, a matrix probe immobilized on the patients temporal bone via a head frame, or similar arrangement. This kind of specialized imaging mode may overcome the shortcomings of ultrasound imaging and stroke diagnosis in the absence of ultrasound contrast agents or other factors (i.e. highly attenuating temporal bone, low sensitivity, poor SNR, probe motion due to the operator, etc.). In some embodiments, such an arrangement may enable a clinician to verify that the clot location as identified by the CT imaging system is further verified via ultrasound imaging, increasing location and treatment region placement accuracy/confidence.

The CT dataset may further provide an indication of the tissue volume that has been compromised with the stroke. This information can also be recorded and identified in the CT dataset, and may be superimposed on the fused CT/ultrasound image, to help provide a predictive treatment outcome value of the STL treatment, or, at least another region of interest to focus the specialized imaging modes on, to detect and monitor recanalization.

When ultrasound therapy is delivered, ultrasound imaging then may be used to determine the magnitude of blood flow to the affected tissue beyond the clot, for example using Doppler. The vessels with visible flow can be matched to vulnerable regions of the brain as segmented (manually, or automatically through some model-based techniques) on the CT image, these vessels may be registered back to the CT image showing color-coded flow into the vulnerable regions. A procedure completion point can be established based on presence or amount of blood flow detected post-therapy. In some embodiments, a CT vessel map may be employed to permit an assessment of recoverable tissue. A user or clinician can set the specific vessel(s) for flow measurement on the CT vessel map, and the registration of the CT image with the ultrasound image may allow Doppler ultrasound to track the blood flow in those vessels. Therapy may be terminated based on the presence or amount of blood flow detected while monitoring the target area with the ultrasound imaging apparatus.

Guided by the clot location, high-resolution/specialized ultrasound imaging modes such as B-mode and Doppler can also be used in a targeted manner to enable treatment monitoring. Treatment monitoring, especially the determination of vessel recanalization, is possible to accomplish in an easier manner, as during the STL treatment, therapy microbubbles will be circulating within the patient's vasculature, which can (incidentally) also be used to enhance the Doppler flow signal during treatment monitoring, even though this would be considered an off-label use in this particular case.

FIG. 7 illustrates one example embodiment of a CT imaging system 700 which may be employed as CT imaging system 310 in FIG. 3. CT imaging system 700 includes a gantry 412 which is capable of rotation about an axis of rotation 714 which extends parallel to the z direction of the system of co-ordinates shown in FIG. 7. To this end, gantry 712 may be driven at a preferably constant, but adjustable speed by a motor 716. On gantry 712 there is mounted a radiation source 718, for example an X-ray source. This X-ray source is connected to a collimator arrangement 720 which, utilizing inter alia a diaphragm arrangement, forms a conical radiation beam 728 from the radiation produced by the radiation source 718, that is, a radiation beam 728 having a finite dimension other than zero in the direction of the z axis as well as in a direction perpendicular thereto (that is, in a plane perpendicular to the axis of rotation 714).

The radiation beam irradiates an examination zone 722 in which an object 724, for example a patient, arranged on a patient table 726, may be situated. The examination zone 722 is shaped as a cylinder whose diameter is determined by the angle of aperture a of the radiation beam 728 (the angle of aperture is to be understood to mean the angle enclosed by a ray of the radiation beam 728 which is situated at the edge in a plane perpendicular to the axis of rotation 714 relative to the plane defined by the radiation source S and the axis of rotation).

After having traversed examination zone 722, X-ray beam 728 is incident on a two-dimensional detector 730 which is attached to gantry 712 and comprises a plurality of detector rows, each of which comprises a plurality of detector elements 731. The detector rows are arranged in planes which are perpendicular to the axis of rotation 714, preferably on an arc of a circle around radiation source 718. However, they may also be formed in a different way; for example, they may describe an arc of a circle around the axis of rotation 714 or be rectilinear. Each detector element 731 that is struck by radiation beam 728 supplies a measuring value for a ray of the radiation beam 728 in each position of the radiation source 718. Sets of such measuring values will also be referred to as projection data sets hereinafter. A projection data set comprises measuring values acquired by one or more detector elements 731 at one or more projection angles.

The X-ray source 718 and the detector 730 together form an acquisition unit. Detector 730 generally is associated with a data storage means (e.g., memory) for storing the acquired projection data. Such storage means may be included in detector 730 or provided as an external separate data storage unit 734 as shown in FIG. 7.

CT imaging system 700 further includes a processing unit 736 for processing the various projection data sets acquired by the acquisition unit to produce a CT imaging dataset. CT imaging system 700 further includes a display unit 738 for displaying reconstructed images or image portions, and imaging mode support for CT angiography, in which a CT contrast agent is used to highlight vasculature and blood flow (or the absence thereof).

Example embodiments of a sonothrombolysis treatment and ultrasound imaging system which may be employed as sonothrombolysis treatment and ultrasound imaging system 320 in FIG. 3 are disclosed in U.S. Patent Application Publication 2010/160779, and in U.S. Provisional Patent Applications 61/842,402, and 61/842,404.

FIG. 8 is a functional block diagram of one embodiment of a sonothrombolysis treatment and ultrasound imaging system 800 which may be employed sonothrombolysis treatment and ultrasound imaging system 320 in FIG. 3. Beneficially, sonothrombolysis treatment and ultrasound imaging system 800 comprises both a sonothrombolysis treatment apparatus and an ultrasound imaging apparatus, integrating ultrasound treatment and ultrasound imaging functions into a single system. In sonothrombolysis treatment and ultrasound imaging system 500, the sonothrombolysis treatment apparatus and ultrasound imaging apparatus share one or more common components, such as a processor, memory, beamformer(s), ultrasound array(s), etc., as described in more detail below. However, in general a sonothrombolysis treatment apparatus and ultrasound imaging apparatus may employ separate componentry.

Sonothrombolysis treatment system 800 includes two transducer arrays 10a and 10b for transmitting ultrasonic waves and receiving echo information. In this example the arrays shown are two dimensional arrays of transducer elements capable of providing 3D image information although an implementation of the present invention may also use one dimensional array of transducer elements which produce 2D (planar) images. Another alternative is to mechanically steer a one-dimensional array to produce the effect of an electronically steered 1D or 2D array. The transducer arrays in this implementation are coupled to microbeamformers 12a and 12b which control transmission and reception of signals by the array elements and in particular the steering and focusing of ultrasonic beams for imaging and therapy. Signals are routed to and from the microbeamformers by a multiplexer 14 by time-interleaving signals. Other implementations may require higher power transmit signals for therapy than those produced by a microbeamformer, in which case transducer drive circuitry capable of higher output power levels may be employed. The multiplexer is coupled to a transmit/receive (T/R) switch 16 which switches between transmission and reception and protects sensitive input circuitry of the main beamformer 20 from high amplitude transmit signals. The transmission of ultrasonic beams from the transducer arrays 10a and 10b under control of the microbeamformers 12a and 12b or other drive circuitry is directed by the transmit controller 18 coupled to the T/R switch, which receives input from the user's operation of the user interface or control panel 38.

The partially beamformed echo signals produced by the microbeamformers 12a, 12b are coupled to a main beamformer 20 where partially beamformed signals from the individual patches of elements are combined into a fully beamformed signal. For example, the main beamformer 20 may have 128 channels, each of which receives a partially beamformed signal from a patch of 12 transducer elements. In this way the signals received by over 1500 transducer elements of a one- or two-dimensional array can contribute efficiently to a single beamformed signal.

The beamformed signals are coupled to a nonlinear echo processor 22. Echo processor 22 acts to separate echo signals arising from tissue structures from those arising from VARs, thus enabling the identification of the strongly nonlinear echo signals returned from VARs. The separated signals are coupled to a signal processor 24 where they may undergo additional enhancement such as speckle removal, signal compounding, and noise elimination.

The processed signals are coupled to a B mode processor 26 and a Doppler processor 28. The structural and motion signals produced by these processors are scan converted and coupled to a volume renderer 34, which produces image data of tissue structure, flow, or a combined image of both characteristics. Volume renderer 34 may convert a 3D data set into a projected 3D image as viewed from a given reference point. This image manipulation is controlled by the user as indicated by the Display Control line between user interface 38 and volume renderer 34. The 2D or 3D images are coupled from the volume renderer to an image processor 30 for further enhancement, buffering and temporary storage for display of static or live 2D MPR or 3D images on an image display 40.

A graphics processor 36 is coupled to the image processor 30 which generates graphic overlays for display with the ultrasound images. These graphic overlays can contain standard identifying information such as patient name, date and time of the image, imaging parameters, and the like, and can also produce a graphic overlay of a therapy beam vector steered by the user as described below. For this purpose the graphics processor receives input from user interface 38. User interface 38 is also coupled to transmit controller 18 to control the generation of ultrasound signals from transducer arrays 10a and 10b in the therapy and imaging modes and hence the images produced by and therapy applied by the transducer arrays. Graphics processor 36 and image processor are associated with one or more memory devices 35 which may store data which is processed by graphics processor 36 and/or image processor 30.

Transducer arrays 10a and 10b transmit ultrasonic waves into the cranium of a patient from one or both sides of the head, although other locations may also or alternately be employed such as the front of the head or the sub-occipital acoustic window at the back of the skull. The sides of the head of most patients advantageously provide suitable acoustic windows for transcranial ultrasound at the temporal bones around and above the ears on either side of the head. In order to transmit and receive echoes through these acoustic windows the transducer arrays must be in good acoustic contact at these locations which may be done by holding the transducer arrays in acoustic coupling contact against the head with a headset.

Sonothrombolysis system 800 may comprise a Vascular Acoustic Resonator (VAR), which operates in combination with the transducer of the system when submitted to the applied ultrasound waves at the required acoustic pressures. Vascular acoustic resonators include any component capable of converting acoustic pressure in a propagation-medium into micron-size displacements, capable of applying strain onto blood clots or vessel walls, also with micron-size deformation amplitude. Examples of suitable VARs include gas-filled microvesicles, i.e. vesicles of nano- or micronic-size comprising a stabilizing envelope containing a suitable gas therein. The formulation and preparation of VARs is well known to those skilled in the art, including, for instance, formulation and preparation of: microbubbles.

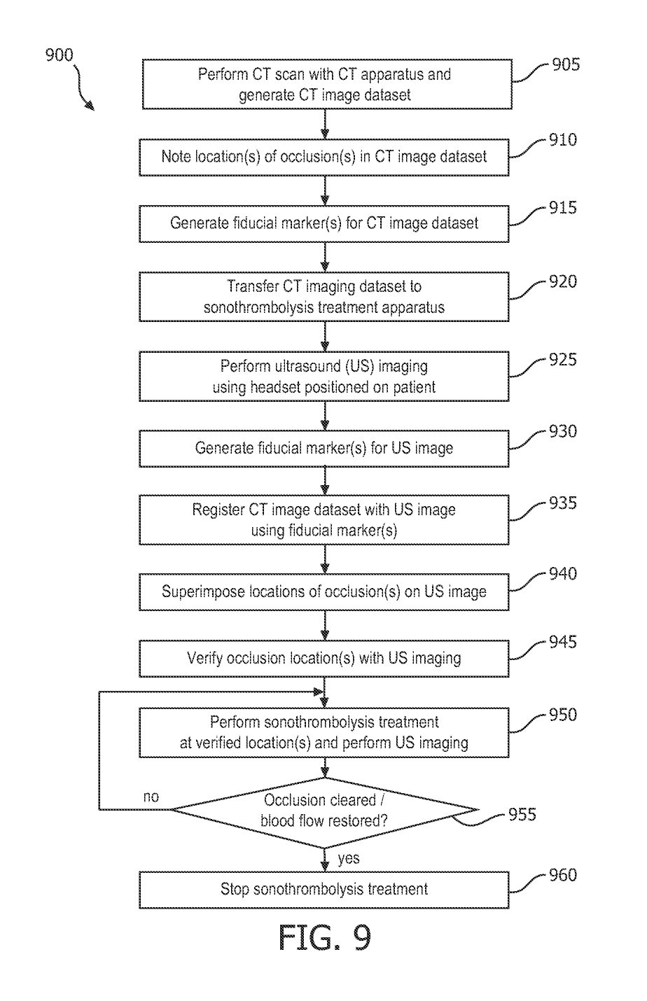

FIG. 9 is a flowchart of one embodiment of a method 900 for sonothrombolysis treatment of a target area, in particular an area of a blood clot causing vascular occlusion.

In an operation 905, a computer tomography (CT) scan is performed by a CT imaging apparatus (e.g., CT imaging system 310 of FIG. 3) for a region of interest in a subject or patient to generate a CT image dataset for the region. For example, the CT image dataset may be a three dimensional (3D) image dataset of the subject's cranium.

In a particular example, the CT scan is performed on a subject's brain to produce a 3D image dataset of the subject's brain which may stored in memory (e.g., memory 314) be used to diagnose the presence of one or more blood clots causing vascular occlusion. A stroke diagnosis may be made on the basis of 3D CT angiogram. If the diagnosis is not an acute ischemic stroke, the patient may be referred elsewhere and the subsequent steps of sonothrombolysis treatment may not be performed. FIG. 1 above shows an example of a CT image 100 revealing a blood clot 110.

In an operation 910, the location(s) of any occlusions or clots are marked or identified in the CT image dataset as explained above.

In some embodiments, operation 910 may be performed via a CT imaging system (e.g., CT imaging system 310 of FIG. 3), and the fiducial markers may be stored with the CT imaging data. For example, in some embodiments, a clinician may observe one or more CT images on a display device (e.g., display 316 in FIG. 3) and may employ a user interface (e.g., mouse, trackball, touch screen, lighten, etc.) and a software algorithm executed by a processor (e.g., processor 312) of the CT imaging system to mark or identify the location(s) of occlusion(s) or clot(s) in the imaged region, and store the marked CT image(s) in memory (e.g., memory 314 in FIG. 3). A marked location identifies a target area for the sonothrombolysis treatment in subsequent operations discussed below. FIG. 4 shows an example of a CT image 400 with a marker 410 indicating the location of a blood clot 110 which will be a target area for sonothrombolysis treatment. In other embodiments the CT imaging data generated by the CT imaging system in operation 905 may be transferred to a separate computer, workstation, or other data processing device, and operation 910 may be performed via that computer, workstation, or other data processing device.

In an operation 915, one or more fiducial markers are generated from a CT image generated in operation 905, and saved with the associated CT imaging data. The fiducial marker(s) for the CT image may be employed in subsequent operations for registering the CT image with an ultrasound image produced in subsequent operation 930. Beneficially, fiducial markers are selected which identify things which are visible in both CT image and the ultrasound image. In various embodiments, fiducial markers may include markers identifying: an outline of at least a portion of the subject's skull bone; a location of the subject's contralateral skull bone; the temporal bone; the brain stem; and one or more corresponding cerebral vessels of the subject. The use of other fiducial markers is contemplated.

In some embodiments, operation 915 may be performed via a CT imaging system (e.g., CT imaging system 310 of FIG. 3), and the fiducial markers may be stored with the CT imaging data. For example, in some embodiments, a clinician may observe one or more CT images on a display device (e.g., display 316 in FIG. 3) of the CT imaging system and may employ a user interface (e.g., mouse, trackball, touch screen, lighten, etc.) and a software algorithm executed by a processor (e.g., processor 312) of the CT imaging system to add one or more fiducial marker(s) to the CT image(s) and store the CT image(s) with the fiducial marker(s) in memory (e.g., memory 314 in FIG. 3). In other embodiments the CT imaging data generated by the CT imaging system in operation 905 may be transferred to a separate computer, workstation, or other data processing device, and operation 915 may be performed via that computer, workstation, or other data processing device.

In an operation 920, the CT image dataset is transferred to a sonothrombolysis treatment apparatus (e.g., sonothrombolysis treatment and ultrasound imaging system 320 of FIG. 3), where it may be stored in memory (e.g., memory 324) and utilized by a processor (e.g., processor 322) of the sonothrombolysis treatment apparatus as described below. Depending on the data input interfaces of the particular sonothrombolysis treatment apparatus, the data may be transferred via wireless link, via a network (e.g., an intranet or internet), via a portable data storage medium such as a DVD or a Flash memory device, etc. In some embodiments, the CT image dataset may be transferred from the CT imaging apparatus to a network server and associated data storage device, and then transferred from the network server to the sonothrombolysis treatment apparatus.

In some embodiments, the order of operations 910, 915 and 920 may be rearranged. That is, for example in some embodiments the CT image dataset may be transferred to the sonothrombolysis treatment apparatus, and the location(s) of any occlusions or clots in the CT image dataset and/or the one or more fiducial marker(s) for the CT imaging dataset may be marked or identified by a clinician or other user via a display device (e.g., display 326 in FIG. 3) and user interface (e.g., mouse, trackball, touch screen, lighten, etc.) associated with the sonothrombolysis treatment apparatus, rather than being generated with the CT imaging system.

In an operation 925, an initial ultrasound imaging scan is performed on the region of interest (e.g., a subject's brain). In some embodiments, the ultrasound imaging is performed by a headset (e.g., headset 500 of FIG. 5) associated with a sonothrombolysis treatment apparatus and which is positioned on the subject's head. The sonothrombolysis treatment apparatus and associated headset may integrate the functions of ultrasound imaging and sonothrombolysis treatment. That is, an ultrasound imaging apparatus employed for ultrasound imaging in operation 925, and a sonothrombolysis treatment apparatus employed for sonothrombolysis treatment in subsequent operations, may be integrated into a single system or unit and may share one or more common components, such as a processor, memory, beamformer(s), ultrasound array(s), etc., examples of which are shown in FIGS. 3 and 8 above.

In an operation 930, one or more fiducial markers are generated from the ultrasound image generated in operation 925. The fiducial marker(s) for the ultrasound image may be employed in subsequent operations for registering the ultrasound image with the CT image dataset and associated fiducial marker(s) produced in operation 915. Beneficially, fiducial markers are selected which identify things which are visible in both CT image(s) and the ultrasound image. In various embodiments, fiducial markers may include markers identifying: an outline of at least a portion of the subject's skull bone; a location of the subject's contralateral skull bone; the temporal bone; the brain stem; and one or more corresponding cerebral vessels of the subject. The use of other fiducial markers is contemplated.

Operation 930 may be performed via a sonothrombolysis treatment and ultrasound imaging system (e.g., sonothrombolysis treatment and ultrasound imaging system 320 of FIG. 3). For example, in some embodiments, a clinician may observe one or more ultrasound images on a display device (e.g., display 326 in FIG. 3) of the sonothrombolysis treatment and ultrasound imaging system and may employ a user interface (e.g., mouse, trackball, touch screen, lighten, etc.) and a software algorithm executed by a processor (e.g., processor 322) of the sonothrombolysis treatment and ultrasound imaging system to add one or more fiducial marker(s) to the ultrasound image(s).

In an operation 9350, the ultrasound image produced in operation 925 is registered or fused with the stored CT image dataset obtained in operations 905 through 915 by means of the fiducial marker(s) generated in operations 915 and 930. By employing the fiducial markers, CT image/ultrasound image registration may thus be limited to dataset translation, rotation, and scaling only, all linear transformations. Finally, a strong boundary condition, such as the approximate location and orientation of the ultrasound probe on the patient's skull can be used as an initial solution for iterative CT/ultrasound image registration algorithms. The implementation of such image registration algorithms would be within the capabilities of those skilled in the art, and further details of such algorithms are not repeated here.

In an operation 940, an ultrasound image is displayed and the location of a target area for sonothrombolysis treatment (e.g., the location of an occlusion or blood clot) which has been identified and marked in the CT image dataset in operation 910 is automatically superimposed on the ultrasound image produced in operation 925 which has been registered with the CT image dataset by means of the fiducial marker(s). In some embodiments the ultrasound image may be fused with a corresponding CT image. Image-fusion may be employed to present an overlay, or a side-by-side registered view of the ultrasound image and the CT image, for example as illustrated in FIG. 6. Image-fusion helps provide a common coordinate system for creating target locations for therapy delivery.

In an operation 945, ultrasound imaging is repeated for the region of interest (e.g., a subject's brain) to verify the location of the target area within the ultrasound image and to adjust one or more parameters of the sonothrombolysis treatment apparatus to cause ultrasound treatment to be directed at the target area. In some embodiments, the size and location of the occlusion(s) or blood clot(s) may be translated into recommended power/energy/time values for therapy delivery.

In an operation 950, the sonothrombolysis treatment apparatus performs the sonothrombolysis treatment of the target area. While the sonothrombolysis treatment is administered, ultrasound imaging may be performed to monitor the target area and provide real time imaging of the target area while the sonothrombolysis treatment is applied to the target area. In some embodiments, high-sensitivity ultrasound imaging modes such as B mode and Doppler imaging are employed for the target area to assess the treatment's progress.

In an operation 955, it is determined by means of the high-sensitivity ultrasound imaging whether or not blood clot or occlusion has been removed and whether blood flow has been restored in the target area. If not, then sonothrombolysis treatment continues in operation 945.

If it is verified that recanalization has occurred, then in an operation 960 sonothrombolysis treatment is stopped. In some embodiments, the sonothrombolysis treatment may be stopped by a user at any point in time, and/or it may be stopped automatically after it has been performed for a predetermined length of time.

While preferred embodiments are disclosed in detail herein, many variations are possible which remain within the concept and scope of the invention. Such variations would become clear to one of ordinary skill in the art after inspection of the specification, drawings and claims herein. The invention therefore is not to be restricted except within the scope of the appended claims.

What is claimed is:

1. A method, comprising:
receiving a computed tomography (CT) image dataset produced by a computed tomography system;
employing an ultrasound imaging apparatus to produce an ultrasound image of a region of a subject including a target area to be treated with sonothrombolysis treatment;
generating one or more fiducial markers for the ultrasound image, wherein the one or more fiducial markers identify a recognizable feature of the subject;
using the one or more fiducial markers for the ultrasound image and one or more corresponding fiducial markers for the CT image dataset to register the ultrasound image with the CT image dataset including a marker identifying the location of the target area in the CT image dataset;
superimposing the marker identifying the location of the target area in the CT imaging dataset with the ultrasound image; and verifying the location of the target area with the ultrasound imaging apparatus.

2. The method of claim 1, wherein the region of the subject includes at least a portion of the subject's head, and wherein the target area corresponds to an area of an occlusion in the subject's brain.

3. The method of claim 2, wherein the one or more fiducial markers include one or more markers identifying at least one of: an outline of at least a portion of the subject's skull bone, a location of the subject's contralateral skull bone, a location of the subject's brain stem, a location of the subject's temporal bone, and one or more corresponding cerebral vessels of the subject.

4. (canceled)

5. (canceled)

6. (canceled)

7. The method of claim 14, further comprising using one or more processors to ascertain one or more values for one or more corresponding imaging parameters of the ultrasound imaging apparatus and treatment parameters of sonothrombolysis treatment based on the location of the target area, so as to obtain desired imaging of the target area.

8. The method of claim 41, further comprising employing the ultrasound imaging apparatus to provide real time imaging of the target area using a Doppler based algorithm to process signals received by the ultrasound imaging apparatus from the target area.

9. (canceled)

10. The method of claim 1, further comprising generating the one or more fiducial markers for the CT image dataset.

11. A system, comprising:
a sonothrombolysis treatment apparatus; and
an ultrasound imaging apparatus,
wherein the system includes one or more processors configured to:
control the ultrasound imaging apparatus to produce an ultrasound image of a region of a subject,
identify in the ultrasound image a location of a target area via a marker for the target area produced from a computed tomography (CT) image dataset, and
control the sonothrombolysis treatment apparatus to provide sonothrombolysis treatment to the target area while controlling the ultrasound imaging apparatus to image the target area.

12. The system of claim 11, wherein the sonothrombolysis treatment apparatus includes a headset including at least one ultrasound transducer array configured to supply ultrasound treatment to an adjustable treatment region and to image the treatment region, and wherein controlling the sonothrombolysis treatment apparatus to provide sonothrombolysis treatment to the target area includes automatically adjusting at least one of a position and an orientation of the ultrasound transducer array to cause the treatment region to match the target area.

13. The system of claim 11, wherein the one or more processors are configured to ascertain one or more values for one or more imaging parameters of the ultrasound imaging apparatus and treatment parameters of the sonothrombolysis treatment apparatus based on the location of the target area so as to obtain at least one of a desired imaging and a desired treatment of the target area.

14. The system of claim 13, wherein the one or more processors are configured to automatically adjust at least one of the one or more imaging parameters of the ultrasound imaging apparatus and the treatment parameters of the sonothrombolysis treatment apparatus based to have the one or more ascertained values.

15. The system of claim 13, wherein the one or more processors are configured to indicate to a user of the system the one or more determined values for the one or more corresponding imaging parameters of the ultrasound imaging apparatus.

16. The system of claim 11, wherein the ultrasound imaging apparatus includes a Doppler mode for processing images of the target area.

17. A method, comprising:
employing an ultrasound imaging apparatus to produce an ultrasound image of a region of a subject;
identifying in the ultrasound image a location of a target area via a marker of the target area produced from a computed tomography (CT) image dataset; and
verifying the location of the target area with the ultrasound imaging apparatus.

18. (canceled)

19. The method of claim 17, further comprising:
automatically ascertaining one or more values for at least one of: one or more imaging parameters of the ultrasound imaging apparatus and one or more treatment parameters based on the location of the target area so as to obtain desired imaging of the target area; and
adjusting at least one of the one or more imaging parameters and one or more treatment parameters to have the one or more ascertained values.

20. (canceled)

21. The system of claim 11, wherein the processor is further configured to identify in the ultrasound image a location of a target area via a marker for the target area produced from a computed tomography (CT) image dataset by:
employing one or more fiducial markers for the ultrasound image, and one or more corresponding fiducial markers for the CT image dataset, to register the ultrasound image with the CT image dataset including a marker identifying the location of the target area in the CT image dataset; and
superimposing the marker identifying the location of the target area in the CT imaging dataset with the ultrasound image.
